(19) United States
(12) Patent Application Publication
(10) Pub. No.: US 2025/0249248 A1
(43) Pub. Date: Aug. 7, 2025

(54) VAGUS NERVE NEUROMODULATION FOR THE TREATMENT OF THE HYPOGLYCEMIC STATE

(71) Applicant: RESHAPE LIFESCIENCES, INC., Irvine, CA (US)

(72) Inventors: Jonathan J. WAATAJA, Plymouth, MN (US); Rajesh K. NIHALANI, Irvine, CA (US)

(21) Appl. No.: 18/702,356

(22) PCT Filed: Nov. 23, 2022
(86) PCT No.: PCT/US2022/080410

§ 371 Date: Jul. 18, 2024

Related U.S. Application Data
(60) Provisional application No. 63/282,397, filed on Nov. 23, 2021.

(51) Int. Cl. A61N 1/36

(52) U.S. Cl. CPC A61N 1/36053; A61N 1/3606; A61N 1/36139

(57) ABSTRACT

Neuroregulation systems and methods for treatment or control of hypoglycemia are provided. In one example, a method of treating hypoglycemia in a subject comprises: applying a first electrical signal to a first nerve or organ of the subject using a neuroregulation system, wherein the first electrical signal initiates a neural stimulation or a neural block on the first nerve or organ of the subject; and optionally applying a second electrical signal to a second nerve or organ of the subject, wherein the second electrical signal initiates a neural stimulation or a neural block on the second nerve or organ of the subject.

### CROSS-REFERENCE TO RELATED APPLICATION

This application is being filed on Nov. 23, 2022, as a PCT International Patent Application and claims priority to and the benefit of U.S. Provisional Application No. 63/282,397, filed Nov. 23, 2021, which is incorporated by reference herein in its entirety.

### INTRODUCTION

Severe hypoglycemia (need for a 3^{rd} party assistance) has an annual incidence of 1.0-1.7 episodes per patient per year. Hypoglycemia can cause loss of consciousness, stroke, coma or death. Repeated hypoglycemic episodes have been linked to cardiovascular disease. Insulin-therapy-induced hypoglycemia (low plasma glucose (PG) typically below 70 mg/Dl) is a problem for diabetics. Repeated hypoglycemic episodes have been linked to cardiovascular disease and severe hypoglycemia (PG below about 54 mg/Dl, which requires need for 3^{rd} party assistance) can cause loss of consciousness, stroke, coma and death.

There are about 235,000 emergency department visits/year to treat diabetic hypoglycemia that cost the health care system is about $120 M/yr. Severe nocturnal hypoglycemia is suspected to contribute to an estimated 6% of all deaths in patients with diabetes below 40 years of age, which can lead to high levels of anxiety. Recent reports indicate that 10% of deaths of patients with Type 1 diabetes were caused by hypoglycemia. For diabetics hypoglycemia primarily results from diabetic medications such as sulfonylureas and more commonly insulin treatment. Insulin-therapy-induced hypoglycemia occurs in both type 1 and type 2 diabetics.

With the increasing proportion of the population suffering from Type 2 diabetes mellitus (T2DM), hypoglycemia is becoming a problem in this diabetic segment. Insulin therapy in Type 2 diabetes is mainly prescribed at the later stages of the disease (HbA1c about 9% or greater). This segment of T2DM population is large and growing with a totaling of 80 million patients worldwide. Henderson et al reported that 73% of insulin dependent T2DM subjects experience hypoglycemic episodes each year and 15% have severe episodes (Henderson, 2003).

Treatments for hypoglycemia typically involve injection of dextrose or glucagon and/or consumption of a fast-acting carbohydrate. However, these treatments are not ideal for nocturnal hypoglycemia and/or contraindicated for severe hypoglycemic episodes. Insulin pump therapy in conjunction with glucose sensor technology decreases the risk of hypoglycemia, but still remains a meaningful problem (Guzman, 2020; Al Hayek, 2018). Less than 1% of insulin dependent diabetics use insulin pumps with issues of maintenance and tolerance required by the continuous use of an external device (Schade, 2006; Walsh, 2015; Bonfanti, 2016).

In type 1 diabetics insulin therapy is required throughout life. About 30 million type 1 diabetic patients worldwide require insulin (Garg, Rewers, & Akturk, 2018). The average individual with type 1 diabetes experiences about two episodes of symptomatic hypoglycemia per week. Severe hypoglycemia has an annual prevalence of 30-40% and an annual incidence of 1.0-1.7 episodes per patient per year (McCrimmon & Sherwin).

With the increasing amount of the population suffering from Type 2 diabetes mellitus (T2DM) hypoglycemic is becoming a problem in this diabetic segment. Insulin therapy in type 2 is mainly prescribed at the later stages of the disease (HbA1c˜9% or greater). This segment of the type 2 diabetic population is large and growing with a totaling of 80 million patients worldwide (Garg et al., 2018). In a study by Henderson et al 73% of insulin dependent T2DM subjects experience hypoglycemic episodes each year and 15% have severe episodes (Henderson, Allen, Deary, & Frier, 2003).

Treatments typically involve consumption of fast acting carbohydrates, injection of glucagon or nasal inhalation of glucagon powder. However, these treatments are not ideal especially for severe hypoglycemic episodes and there is a need for new therapeutic options. Therefore, there is need for new systems and methods for treatment of hypoglycemia.

### Vagus Nerve Neuromodulation for the Treatment of the Hypoglycemic State

In some aspects, the present disclosure provides systems and methods for hypoglycemia vagal nerve stimulation (HVNS). In particular embodiments, the present HVNS system comprises an implantable pulse generator (IPG) in a closed loop with a continuous glucose monitor (CGM), stimulation electrodes/leads attachable to posterior vagus nerve (PVN) cranial to the celiac branch, a programmer to alter settings for therapeutic customization.

In some aspects, the present disclosure also provides a minimally invasive electrode implantation method. In particular embodiments, the present method includes implanting electrodes in a subject to be treated using a less invasive laparoscopic technique for optimal electrode placement with enhanced visualization of the posterior vagus nerve and celiac branch. This can be achieved by reliably locating the celiac branch laparoscopically for correct electrode placement on the PVN.

In some aspects, the present disclosure provides various operating parameters for HVNS. In particular embodiments, implementation of the present method using selected operating parameters is effective to increase plasma glucose by at least about 20 mg/dL within about 30 min after treatment in a subject from a controlled clamped glucose level of 50 mg/dL.

In some aspects, the present disclosure provides the safety of stimulation on vagal nerve and end organs. From animal studies presented in the Examples of this disclosure, little-to-no adverse behavior or organ damage is observed as a result of stimulation or gross necropsy.

In some aspects, a system for treating hypoglycemia in a subject comprises: (1) at least one electrode adapted to be placed on and deliver electrical signal to a posterior vagus nerve (PVN) of the subject or the celiac vagus nerve branch of the PVN; (2) an implantable pulse generator operably connected to the at least one electrode, wherein the implantable pulse generator comprises a power module and a programmable therapy delivery module, wherein the programmable therapy delivery module is configured to deliver at least one therapy program, wherein the at least one therapy program comprises at least one electrical signal treatment applied to the PVN through the at least one electrode, (3) an external component comprising a communication system and a programmable storage and communication module, wherein programmable storage and communication module are configured to store the at least one therapy program and to communicate the at least one therapy program to the implantable pulse generator, and (4) a glucose sensor operably connected to the implantable pulse generator and the external component, wherein the glucose sensor is configured to continuously monitor plasma glucose of the subject and to detect an increase or decrease of plasma glucose from a pre-determined threshold level, wherein, the implantable pulse generator is triggered to deliver the at least one electrical signal treatment when the plasma glucose of the subject is of or below a first pre-determined threshold, wherein the implantable pulse generator ceases to deliver the at least one electrical signal treatment when the plasma glucose of the subject is of or above a second pre-determined threshold, wherein the at least one electrical signal treatment comprises an electrical signal pattern having a frequency from about 1 Hz to about 200 Hz, a pulse width from about 0.1 microseconds (ms) to about 10 ms in about 0.1 ms steps, a pulse amplitude from about 0.1 mA to about 12 mA in about 0.1 mA steps, and wherein the electrical signal treatment is configured to initiate neural stimulation on PVN of the subject.

In some embodiments, a method of treating hypoglycemia in a subject comprises: applying the at least one electrical signal treatment to a posterior vagus nerve (PVN) of a subject or the celiac vagus nerve branch of the PVN of the subject using the present system.

In another example, a system for treating hypoglycemia in a subject comprises: (1) a first electrode adapted to be placed on and deliver electrical signal to a first nerve or organ; (2) optionally a second electrode adapted to be placed on and deliver electrical signal to a second nerve or organ; (3) an implantable pulse generator operably connected to the first and/or the second electrode, wherein the implantable pulse generator comprises a power module and a programmable therapy delivery module, wherein the programmable therapy delivery module is configured to deliver at least one therapy program comprising a first therapy program and optionally a second therapy program, wherein the first therapy program comprises a first electrical signal treatment applied to the first nerve or organ through the first electrode, wherein the second therapy program comprises a second electrical signal treatment applied to the second nerve or organ through the second electrode, and wherein the first and/or the second electrical signal are each configured to initiate activity on the first and/or the second nerve or organ respectively, and wherein the activity is a neural stimulation or a neural block; and (4) an external component comprising a communication system and a programmable storage and communication module, wherein programmable storage and communication module are configured to store the at least one therapy program and to communicate the at least one therapy program to the implantable pulse generator.

In some embodiments, a method of treating hypoglycemia in a subject, the method comprising: (1) applying a first electrical signal to a first nerve or organ of the subject using a system as described herein, wherein the first electrical signal initiates a neural stimulation or a neural block; and (2) optionally applying a second electrical signal to a second nerve or organ of the subject using the system, wherein the second electrical signal initiates a neural stimulation or a neural block.

In some embodiments, the first and/or the second electrical signal are each independently configured to upregulate or downregulate activity respectively on the first and/or second target nerve or organ. In some embodiments, the first and the second electrical signals are applied concurrently, or simultaneously, or intermittently, or during substantially the same times, or during substantially different times, or in a coordinated fashion. In some embodiments, the first and/or the second electrical signal treatments are each continuously applied to the first target nerve or organ and/or the second target nerve or organ respectively. In certain embodiments, the first electrical signal is an upregulation or stimulation signal.

In some embodiments, the method further comprises a glucose sensor configured to continuously monitor plasma glucose of the subject, wherein the glucose sensor is operably connected to the implantable pulse generator and the external component. In some embodiments, the glucose sensor is configured to detect an increase or decrease of plasma glucose from a pre-determined threshold level. In some embodiments, the implantable pulse generator is triggered to deliver the first and/or the second electrical treatment when the plasma glucose of the subject is of or below a first pre-determined threshold, and wherein the implantable pulse generator ceases to deliver the first and/or the second electrical treatment when the plasma glucose of the subject is of or above a second pre-determined threshold.

In some embodiments, the first nerve or organ and the second nerve or organ are each independently selected from the group consisting of the vagus nerve, anterior vagus nerve, posterior vagus nerve, hiatus on posterior nerve, hepatic branch of vagus nerve, celiac branch of vagus nerve, splanchnic nerve, renal nerve, renal artery, sympathetic nerves, baroreceptors, glossopharyngeal nerve, duodenum, jejunum, ileum, small bowel, colon, stomach, esophagus, liver, spleen, pancreas, and combinations thereof. In particular embodiments, the first nerve or organ is celiac branch of posterior vagus nerve.

In some embodiments, the method further comprises a glucose sensor configured to continuously monitor plasma glucose of the subject having the condition of Type-1 or Type-2 diabetes, wherein the glucose sensor is operably connected to the implantable pulse generator and the external component. In at least these example embodiments, the glucose sensor is configured to detect an increase or decrease of plasma glucose from a pre-determined threshold level. In related embodiments, the implantable pulse generator is triggered to deliver the first and/or the second electrical treatment when the plasma glucose of the subject is of or below a first pre-determined threshold, and wherein the implantable pulse generator ceases to deliver the first and/or the second electrical treatment when the plasma glucose of the subject is of or above a second pre-determined threshold.

Similar to the embodiments described above, the subject having the condition of Type-1 or Type-2 diabetes can be treated where the first nerve or organ and the second nerve or organ are each independently selected from the group consisting of the vagus nerve, anterior vagus nerve, posterior vagus nerve, hiatus on posterior nerve, hepatic branch of vagus nerve, celiac branch of vagus nerve, splanchnic nerve, renal nerve, renal artery, sympathetic nerves, baroreceptors, glossopharyngeal nerve, duodenum, jejunum, ileum, small bowel, colon, stomach, esophagus, liver, spleen, pancreas, and combinations thereof. In particular embodiments, the first nerve or organ is celiac branch of posterior vagus nerve.

In some embodiments, the first nerve or organ and the second nerve or organ are different. In some embodiments, the first electrical signal is applied on a hepatic branch of a vagus nerve or an anterior vagus nerve central to a branching point of a hepatic nerve. In some embodiments, the first electrical signal is applied on a celiac branch of a vagus nerve, or an anterior vagus nerve central to a branching point of a celiac nerve, or liver, pancreas, or both.

In some embodiments, the first and/or the second electrical signals each have an on time and an off time, wherein the off time is selected to allow at least a partial recovery of the activity of the first and/or the second nerve or organ. In some embodiments, the on time is configured to commence upon the detection of plasma glucose level of or below about 50 mg/dL, of or below about 60 mg/dL, of or below about 70 mg/dL, of or below about 80 mg/dL. In some embodiments, the first and/or the second electrical signal treatment are configured to increase the plasma glucose level by at least about 5 mg/dL in about 10 minutes. In some embodiments, the first and/or the second electrical signal treatment are configured to increase the plasma glucose level by at least about 10 mg/dL in about 20 minutes. In some embodiments, the first and/or the second electrical signal treatment are configured to increase the plasma glucose level by at least about 20 mg/dL in about 30 minutes.

In some embodiments, the first electrical signal has a frequency of about 0.01 Hz to about 200 Hz, from about 0.1 Hz to about 100 Hz, or from about 1 Hz to about 20 Hz. In other embodiments, the first electrical signal has a frequency of about 200 Hz to about 10 k Hz.

In some embodiments, the second electrical signal has a frequency of about 0.01 Hz to about 200 Hz, from about 0.1 Hz to about 100 Hz, or from about 1 Hz to about 20 Hz. In other embodiments, the second electrical signal has a frequency of about 200 Hz to about 10 k Hz.

In some embodiments, the first electrical signal has a frequency of about 0.01 Hz to about 200 Hz, and wherein the second electrical signal has a frequency of about 200 Hz to about 10 k Hz.

In some embodiments, the first electrical signal and/or the second electrical signal each independently comprise a signal pattern, wherein each signal pattern comprises a pulse having a pulse width from about 10 microseconds to about 10,000 microseconds.

In other related embodiments, the pulse of the first and/or the second electrical signal is monophasic pulse, or biphasic pulse, or combinations thereof. In some embodiments, the first and/or the second electrical signal each independently have an on time of about 30 seconds to about 30 minutes. In some embodiments, the first and/or the second electrical signal each independently have a current amplitude in a range from about 0.01 mAmps to about 20 mAmps. In some embodiments, the first and/or the second electrical signal each independently comprise an abrupt start of pulses, or a ramp up of current/voltage amplitude, or a ramp up of frequency, or a ramping up of pulse widths, or combination thereof at or near initiation of applying the first and/or the second electrical signal.

In some embodiments, the first and/or the second electrical signal treatments are configured to be applied intermittently multiple times in a day and over multiple days, wherein the first and/or the second electrical signal each have a frequency selected to upregulate activity on the first nerve or organ and has an on time and an off time, wherein the off time is selected to allow at least a partial recovery of the activity of the first nerve or organ.

In some embodiments, the programmable storage and communication module are configured to store and communicate more than one therapy program, wherein each therapy program is different from one another, and is configured to be selected for communication.

In some embodiments, the system further comprises a transmitter operably connected to the glucose sensor, wherein the transmitter is configured to communicate data generated by the glucose sensor to an external communication device.

In some embodiments, the communication system is selected from a group consisting of an antenna, blue tooth technology, radio frequency, Wi-Fi, light, sound and combinations thereof, and wherein the communication system is configured to communicate parameters of the at least one therapy program to an external communication device.

In some embodiments, a method of making a system for treating hypoglycemia in a subject comprises: (1) connecting a first electrode to an implantable pulse generator and placing the first electrode to a first nerve or organ; (2) optionally connecting a second electrode to the implantable pulse generator and placing the second electrode to a second nerve or organ; (3) configuring a programmable therapy delivery module of the implantable pulse generator to deliver at least one therapy program comprising a first electrical signal treatment and optionally a second electrical signal treatment, wherein the first electrical signal treatment is configured to be applied to the first nerve or organ through the first electrode, and the second electrical signal treatment is configured to be applied to the second nerve or organ through the second electrode, and wherein the first and/or the second electrical signal each initiate a neural stimulation or a neural block; and (4) configuring a programmable storage and communication module of an external component to store the at least one therapy program and to communicate the at least one therapy program to the implantable pulse generator.

### Definition and Interpretation of Selected Terms

The term “about” is not intended to either expand or limit the degree of equivalents which may otherwise be afforded a particular value. The term “about” in the context of the present disclosure means a value within 10% (±10%) of the value recited immediately after the term “about,” including any numeric value within this range, the value equal to the upper limit (i.e., +10%) and the value equal to the lower limit (i.e., −10%) of this range. For example, the value “100” encompasses any numeric value that is between 90 and 110, including 90 and 110 (with the exception of “100%,” which always has an upper limit of 100%).

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components, unless context requires otherwise.

“Cycle” as used herein means one repetition of a repetitive pattern of electrical signals.

“Stimulation cycle” particularly refers to low frequency stimulation signal.

“Concurrently” used here in generally means that in situations where multiple electrical signals are applied, in at least one time period, the multiple electrical signals are applied simultaneously or about the same time.

“Duty Cycle” as used herein means the percentage of time charge is delivered to the nerve in one cycle. In embodiments, duty cycle can be modified by decreasing pulse width and/or by adding inactive phases between pulses or both.

“Frequency” as used herein means the reciprocal of the period measured in Hertz.

“High Duty Cycle” as used herein refers to a pattern of electrical signals with a duty cycle of about 76% or greater.

“Low Duty Cycle” as used herein refers to a pattern of signals with a duty cycle of about 75% or less.

“High frequency” as used herein generally refers to a frequency of about 200 Hz or more. “High frequency signal” as used herein generally refers to HFAC or HFAV having a frequency of about 200 Hz or more. High frequency signal is particularly used to downregulate or block nerve activity.

“Low frequency” as used herein generally refers to a frequency of about 200 Hz or less.

“Low frequency signal” or “low frequency stimulation signal” as used herein generally refers to stimulation signal having a frequency of 199 Hz or less. Stimulation signal is particularly used to upregulate or stimulate nerve activity.

“HFAC” as used herein refers to high frequency alternating current.

“HFAV” as used herein refers to high frequency alternating voltage.

“Hz” as used herein refers to Hertz.

“Off Time” as used herein refers to a period when no charge is being delivered to the nerve. In embodiments, off time is on the order of seconds and/or minutes.

“On Time” refers to a period of time in which multiple micro and/or millisecond cycles and/or stimulation cycle and/or stimulation active phase are applied to the nerve. In embodiments, on time is on the order of seconds and/or minutes.

“Period” refers to the length of time of one charge phase and one recharge phase, which can include one or more pulse delays. “Stimulation period” particularly refers to the length of time of one charge phase and one recharge phase in a low frequency stimulation signal. Stimulation period can also include one or more pulse delays.

“Pulse Amplitude” is the height of the pulse in amperes or voltage relative to the baseline.

“Pulse Delay” as used herein refers to an aspect of the period wherein the impedance across a parallel electrical path with the nerve is at or close to 0 Ohms, with the intention of avoiding any unwanted electrical signals being delivered to the nerve.

“Pulse Width” as used herein refers to the length of time of the pulse.

“Ramp Down” as used herein refers to the period at the end of the application of an electrical signal, or between different patterns of electrical signals, to a nerve of a patient where the pulse amplitude of the signal decreases.

“Ramp Up” as used herein refers to increasing the pulse amplitude until the amplitude desired for therapy is reached at the start of an applied electrical signal or between different patterns of electrical signals. The starting amplitude of ramping may be below the current/voltage threshold of blocking.

“Therapy Cycle” as used herein refers to a discrete period of time that contains one or more on times and off times. The pattern of on and off times within the therapy cycle can be repetitive, non-fixed or randomized throughout a therapy schedule.

“Therapy Parameters” as used herein includes, but is not limited to, frequency, pulse width, pulse amplitude, on time, off time and pattern of electrical signals.

“Therapy Schedule” as used herein refers to the time of day when therapy cycles start, the number of therapy cycles, timing of therapy cycles and duration of the delivery of therapy cycles for at least one day of the week.

“Nerve” used herein generally encompasses a nerve or any part thereof, including but not limited to nerve branch, nerve fiber, trunk, branching point.

“Anterior vagus nerve (AVN)” or “anterior vagus trunk” distributes fibers on the anterior surface of the esophagus, and consists primarily of fibers from the left vagus. “Posterior vagus nerve (PVN)” or “posterior vagus trunk” consists primarily of fibers from the right vagal nerve distributed on the posterior surface of the esophagus. Anterior vagus nerve and posterior vagus nerve are two different and separate nerves.

“Hepatic branch” used herein refers to a nerve branch of the anterior vagus nerve below the diaphragm. Hepatic branch encompasses any segment of the anterior vagus nerve cranial to the hepatic branch. In particular, Hepatic branch carries afferent information from the pancreas to the brain and efferent information from the brain to the pancreas.

“Celiac branch” used herein generally refers to a nerve branch of the posterior vagus nerve below the diaphragm. Celiac branch encompasses any segment of the posterior vagus nerve cranial to celiac branch. In particular, celiac branch carries afferent information from the pancreas to the brain and efferent information from the brain to the pancreas.

“Celiac fiber” used herein refers to an afferent or efferent axon that travels within the length of the vagal nerve between the pancreas and the brain. The afferent axon travels from the pancreas through the celiac branch of the vagal nerve where it then travels into the posterior vagus below the level of the diaphragm. The afferent axon next enters the thoracic cavity and primarily into the right cervical segment. The afferent axon then enters the brainstem and form a synaptic connection. The efferent fiber is a part of the parasympathetic nervous system. The preganglionic cell body of the efferent fiber is in the brain stem and travels the length of the vagal nerve (similar to the afferent fiber) to its postganglionic neuron in close proximity to the pancreas.

“Hepatic fiber” used herein refers to an afferent or efferent axon that travels within the length of the vagal nerve between the liver and the brain. The afferent axon travels from the liver through the hepatic branch of the vagal nerve where it then travels into the anterior vagus below the level of the diaphragm. The afferent axon next enters the thoracic cavity and primarily into the left cervical segment. The afferent axon then enters the brainstem and form a synaptic connection. The efferent fiber is a part of the parasympathetic nervous system. The preganglionic cell body of the efferent fiber is in the brain stem and travels the length of the vagal nerve (similar to the afferent fiber) to its postganglionic neuron in close proximity to the liver.

When ranges are provided, the range includes both endpoint numbers as well as all real numbers in between. For example, a range of 200 Hz to 25 kHz includes, for example, 201 to 25 kHz, 202 to 25 kHz, as well as 24,999 Hz to 200 Hz, 24,998 Hz to 200 Hz, and 201 Hz to 24,999 Hz, 202 Hz to 24,998 Hz.

With reference now to the various drawing figures in which identical elements are numbered identically throughout, a description of embodiments of the present disclosure will now be described.

### BRIEF DESCRIPTION OF THE DRAWINGS

FIG. 1. is a graphical illustration of the change in blood glucose over time with stimulation of the celiac branch of the vagus nerve in type 2 diabetic Zucker rats.

FIG. 2 shows a schematic of system in which an implantable glucose sensor communicates with a pulse generator to initiate vagus nerve stimulation.

FIG. 3 shows a schematic of system in which an implantable glucose sensor communicates first with an external device attached to the outside of the skin which then communicates with the pulse generator to initiate vagus nerve stimulation.

FIG. 4: Anatomy of the vagus nerve indicating branching points of interest and what is meant by cranial to a branching point.

FIG. 5: Example of a small IPG positioned on the vagus nerve cranial to the celiac branching point in the abdominal cavity.

FIG. 6: Example of a small IPG positioned on the vagus nerve cranial to the celiac branching point in the cervical cavity.

FIG. 7: Example of a small IPG positioned on the vagus nerve cranial to the celiac branching point in the abdominal cavity.

### DETAILED DESCRIPTION

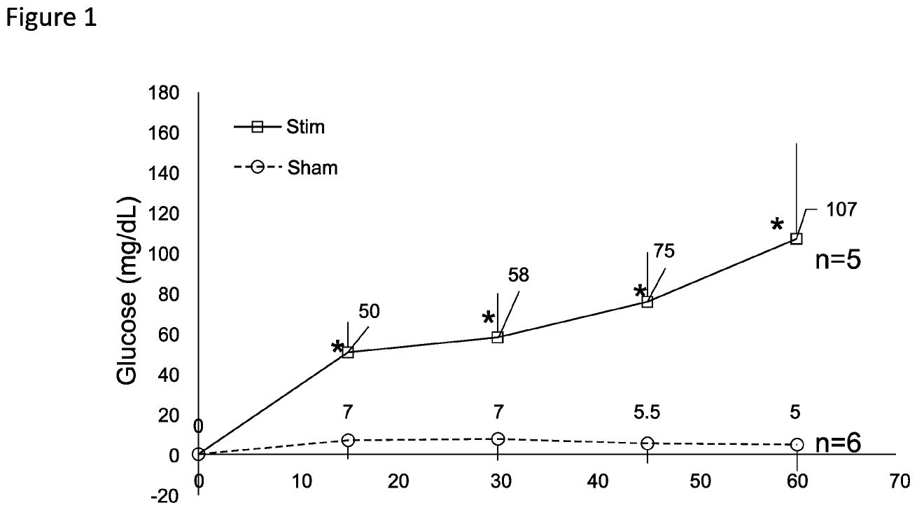

Referring now to FIG. 1, where data was obtain using T2DM Zucker rat model which demonstrated that stimulation alone (1 Hz 1 mA, 4 ms pulse width) of the vagus nerve celiac branch, or posterior vagal trunk above the branching point of the celiac, causes a significant increase in plasma glucose of 42 mg/dL by 15 min. In some embodiments this stimulation is intended to release glucagon from alpha cells of the pancreas to modulate blood glucose. While not wanting to be bound by any particular theory, the ability of a continuous or intermittent signal to initiate the increase in plasma glucose is contemplated. In at least these example embodiments, the system may include parameters that only provide stimulation to the target nerve. In related embodiments, stimulation and block may be used in combination to allow for consistent treatment of elevated and lowered blood glucose levels in a subject.

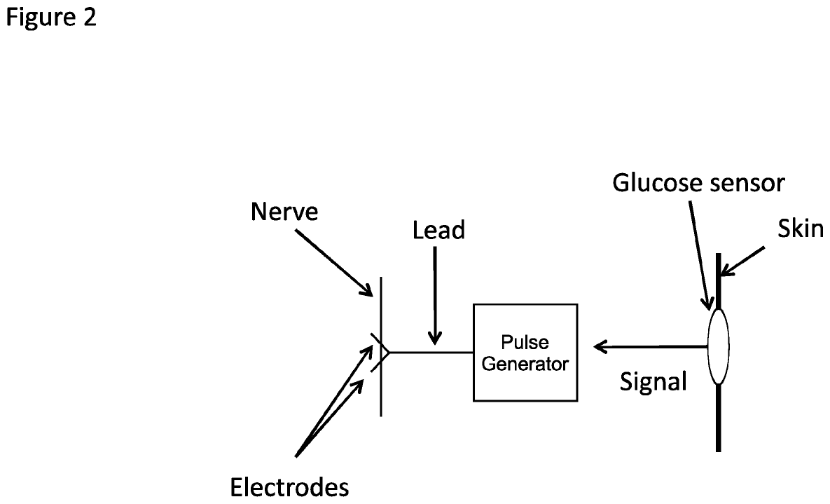

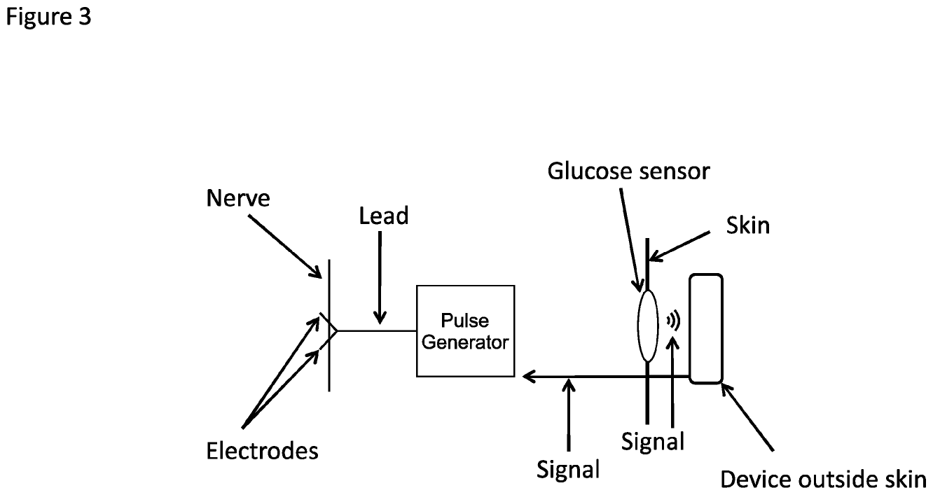

Referring to FIGS. 2-3, wherein the HVNS system would include a pulse generator, leads that are placed on the vagus nerve and an implantable glucose sensor (to monitor plasma glucose levels). The sensor sampling rate would be from about 1 second to 10 min. FIG. 2 shows a schematic of system in which an implantable glucose sensor communicates with a pulse generator to initiate vagus nerve stimulation. The implantable sensor would detect low plasma glucose levels and send a signal to turn the pulse generator on. FIG. 3 shows a schematic of system in which an implantable glucose sensor communicates first with an external device attached to the outside of the skin which then communicates with the pulse generator to initiate vagus nerve stimulation.

The communication between the pulse generator and the glucose sensor can be through, but not limited to, blue tooth technology, radio frequency, Wi-Fi, light or sound. In some embodiments the glucose sensor would be below the layer of the skin and communicate to a device outside of the skin with a battery to power wireless communication. The communication between the glucose sensor and the device outside the body can be through, but not limited to, blue tooth technology, radio frequency, Wi-Fi, light or sound. The device outside of the skin would then communicate with the pulse generator through, but not limited to, blue tooth technology, radio frequency, Wi-Fi, light or sound. The implantable glucose sensor, or the external device that communicates with the implantable glucose sensor, could also communicate with a smart device (such as a phone running an app) to display plasma glucose levels and send an alarm when plasma glucose reaches an unsafe low level. The communication to the smart device can be through, but not limited to, blue tooth technology, radio frequency, Wi-Fi, light or sound. Stimulation parameters include a frequency range between 0.01 Hz to 200 Hz, current or voltage amplitude range: 0.1 mA to 12 mA or 0.1 to 12 volts, pulse width range: 0.1 ms to 10 ms. Stimulation can be continuous or bursting with inter-burst intervals ranging from milliseconds, seconds to minuets.

Site of stimulation include any segment of the vagus nerve. This includes sub-diaphragmatic anterior or posterior vagus trunks and branches of the sub-diaphragmatic vagal trunks such as the celiac branch originating from the posterior vagus trunk, the accessory celiac branch, originating from the anterior vagus trunk or the hepatic branch, originating from the anterior vagus trunk. Sites of stimulation also include the anterior or posterior thoracic vagus, or the left or right cervical vagus. Any combination of vagus nerve stimulation sites is included.

In other embodiments, the HVNS system is entirely closed looped with the primary cell RNR incorporating blue-tooth capability to directly communicate with the glucose transmitter. Low duty cycle on demand stimulation may facilitate use of a small primary cell device without the need for recharging. The CGM transmitter may communicate with a smart device allowing physicians to optimize therapy parameters during a controlled type-2 diabetes trial.

In at least one example embodiment, a system to treat hypoglycemia would include a pulse generator, leads that are placed on the vagus nerve and an implantable glucose sensor. The sensor sampling rate would be from about 1 second to 10 min. The implantable sensor would detect low blood glucose levels and send a signal to turn the pulse generator on (see FIG. 2). The communication between the pulse generator and the glucose sensor can be through, but not limited to, Bluetooth technology, radio frequency, Wi-Fi, light or sound. In some embodiments the glucose sensor would be below the layer of the skin and communicate to a device outside of the skin with a battery to power wireless communication (FIG. 3). The communication between the glucose sensor and the device outside the body can be through, but not limited to, blue tooth technology, radio frequency, Wi-Fi, light or sound. The device outside of the skin would then communicate with the pulse generator through, but not limited to, Bluetooth technology, radio frequency, Wi-Fi, light or sound. The implantable glucose sensor, or the external device that communicates with the implantable glucose sensor, could also communicate with a smart device to display blood glucose levels and send an alarm if stimulation is about to initiate. The communication to the smart device can be through, but not limited to, blue tooth technology, radio frequency, Wi-Fi, light or sound. Stimulation parameters include: frequency range: 0.01 Hz to 200 Hz, current or voltage amplitude range: 0.1 mA to 12 mA or 0.1 to 12 volts, pulse width range: 0.1 ms to 10 ms. Stimulation can be continuous or bursting with inter-burst intervals ranging from milliseconds, seconds to minuets.

In some embodiments one or more, or any combination, of stimulation parameters (frequency, current/voltage amplitude, pulse width or a bursting pattern) can change with different glucose levels. An example would be to have a lower frequency if blood glucose falls into a less severe hypoglycemic state (such as 65 mg/dL). The stimulation frequency here could be, but not limited to, a low frequency, such 0.1-2 Hz. However, if glucose falls into a more severe level (such as 50 mg/dL) the device output would be, but not limited to, a higher frequency such as greater than 2 Hz or more (example 3-60 Hz). An example of a combination of output parameters changing between different glucose levels would be to apply a lower frequency signal, such as, but not limited to 0.1 to 2 Hz and a low current amplitude, such as, but not limited to, 0.1-2 mA (or this rang in volts), when the sensor detects a minor hypoglycemic event (such as, but not limited to 65 mg/dL). However, if the sensor detects a more severe hypoglycemic episode (such as, but not limited to 50 mg/dL) the frequency would be, but not limited to, higher such as 3-60 Hz combined with the combination of a higher, but not limited to, current amplitude output such as >2 mA and <12 mA (or this range in volts). Any combination of different stimulation parameters could be used dependent on the reading from the level of glucose sensor.

Stimulation parameters (frequency, current/voltage amplitude, pulse width or a bursting pattern) can also change during the course of recovery from hypoglycemia. Such as a higher frequency output (example 3-60 Hz) when glucose falls to a severe level (such as 50 mg/dL). The frequency would then gradually decreases during the course of recovery from hypoglycemia and signal cessation when glucose is restored to a safe level. The change in stimulation frequency during recovery could be a function based on a change in frequency vs time such as, but not limited to linear, exponential or logarithmic. For example, if a patient reaches a severe hypoglycemic state (such as 50 mg/dL) the frequency could be high (3-60 Hz) and then decrease in a linear (or non-linear) fashion over time until glucose is at a safe level (above 70 mg/dL). The change in frequency may also be dependent on the glucose level or the rate of change of the glucose levels during recovery. For example the frequency could decrease by 2 Hz for every increase in glucose of 2 mg/dL. The change in signal output could be any combination of signal parameters during the recovery from hypoglycemia.

In some embodiments there can be cyclical on and off times during the episodic stimulation period. The on times could range from 1 sec-5 min and off times range from 1 sec-5 min.

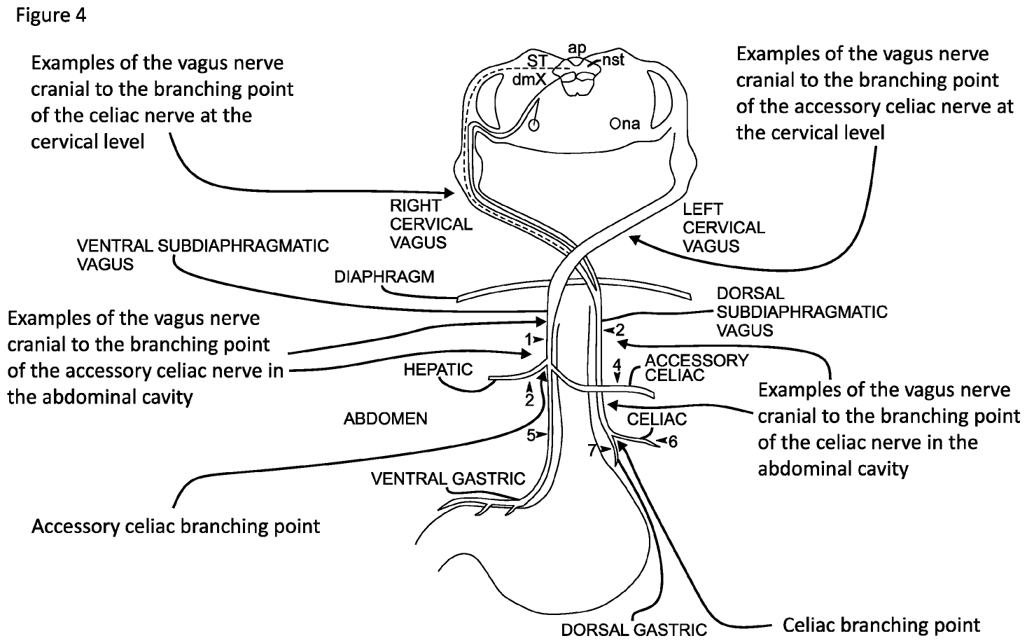

Referring now to FIG. 4 which shows the site of stimulation includes any segment of the vagus nerve. These sites include the celiac branch originating from the posterior vagal trunk, the accessory celiac branch, originating from the anterior vagal trunk or the hepatic branch, originating from the anterior vagal trunk. Sites of stimulation may also include any segment of the vagus nerve cranial to the branching points of the celiac nerve or the accessory celiac nerve. There may also be multiple stimulation sites on the vagus nerve.

Stimulation for a hypoglycemia treatment may be infrequent (examples: 2 stimulation episode/week, 1 stimulation episode/1 week, 1 stimulation episode/2 weeks, 1 stimulation episode/month, 2 stimulation episodes/year or 1 stimulation episode/year) for short stimulation episode durations (examples: 1 min, 5 min & 30 min), at low frequencies (examples: 1 Hz, 5 Hz & 10 Hz), pulse widths between 0.1 ms to 10 ms and current or voltage amplitude range: 0.1 mA to 12 mA or 0.1 to 12 volts. This would consume little, in terms of energy expended on the order of a week, month or year, and allow for a small battery and small implantable pulse generator (IPG or micro-neuroregulator).

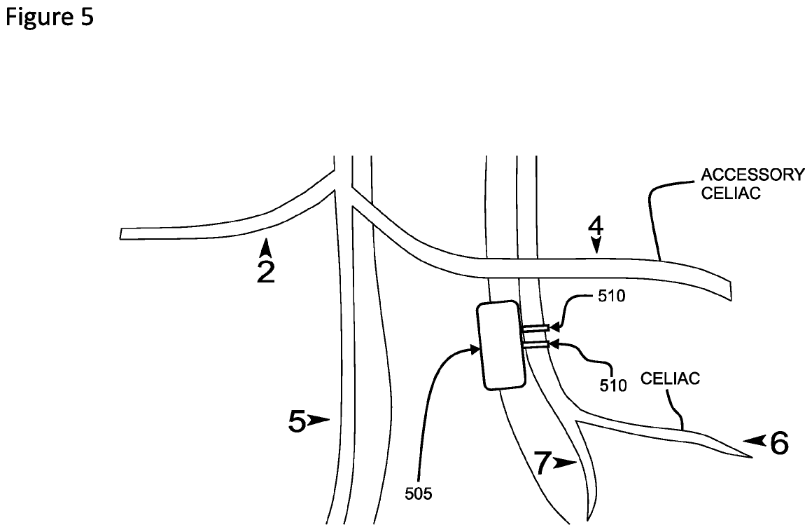

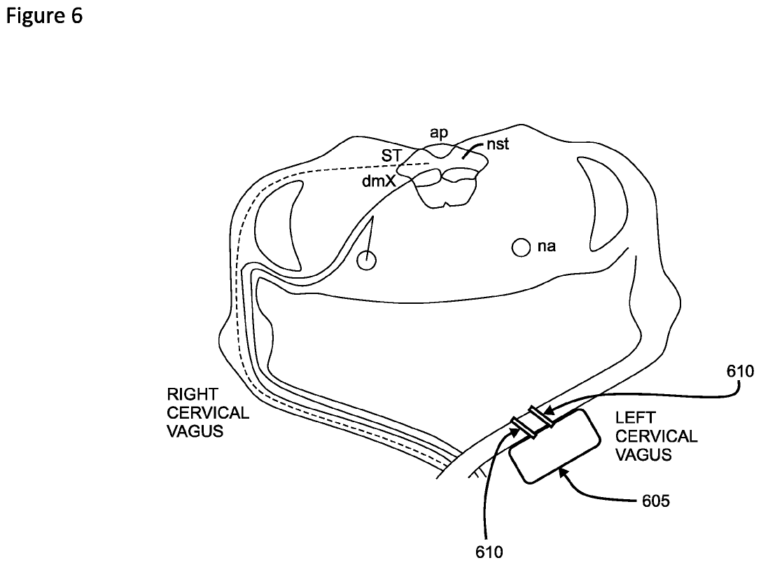

Referring now to FIGS. 5-6 where a small IPG (505, 605) may be positioned directly on the nerve with no need for leads and a less complicated implant procedure. Conductive electrodes (610) which deliver stimulation pulses to the nerve and would be positioned directly on the IPG (FIG. 6). The implanted pulse generator (505, 605) could be placed on the celiac branch of the vagus nerve, the accessory celiac branch or any segment of the vagus nerve cranial to the branching point of the celiac or accessory celiac nerves. This may include the right and/or left cervical aspect of the vagus nerve. In some embodiments the IPG that is positioned on the nerve would be anchored to an adjacent anatomical feature such as the esophagus with the intent to reduce movement of the IPG (505, 605) on the nerve.

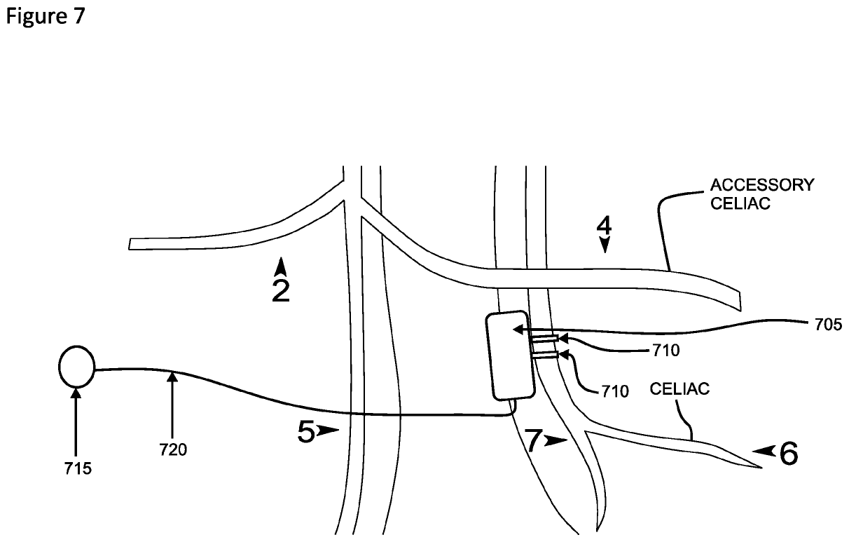

Referring now to FIG. 7, where the micro-neuroregulator/IPG (705) would be connected with a wire (720) to a Micro Subcutaneous Wireless charger (715) below the layer of the skin. A signal from outside of the body, such as a radio frequency signal, light, or sound, would be used to periodically charge the device. The IPG (705) positioned on the nerve, via electrode(s) (710), may be a non-rechargeable primary cell device or a rechargeable device. The method of charging may be delivery of a radio frequency (RF) signal with a coil positioned above the layer of the skin and sending energy to a Micro Subcutaneous Wireless Charger.

Hypoglycemia is not only observed in diabetics but also arises from other diseases such as, but not limited to, kidney failure, certain tumors, liver disease, hypothyroidism, inborn errors of metabolism, severe infections, reactive hypoglycemia, and a number of drugs including alcohol use. The proposed device may help treat hypoglycemia in patients with these medical conditions.

### REFERENCES

Garg, S. K., Rewers, A. H., & Akturk, H. K. 2018. Ever-Increasing Insulin-Requiring Patients Globally. Diabetes Technol Ther, 20(S2): S21-S24.
Henderson, J. N., Allen, K. V., Deary, I. J., & Frier, B. M. 2003. Hypoglycaemia in insulin-treated Type 2 diabetes: frequency, symptoms and impaired awareness. Diabet Med, 20(12): 1016-1021.
McCrimmon, R. J., & Sherwin, R. S. Hypoglycemia in type 1 diabetes. Diabetes, 59(10): 2333-2339.

The above specification, examples and data provide a complete description of the manufacture and use of the composition of the invention. Since many embodiments of the invention can be made without departing from the spirit and scope of the invention, the invention resides in the claims hereinafter appended.

The following numbered clauses define further example aspects and features of the present disclosure:

1. A system for treating hypoglycemia in a subject, the system comprising:
a first electrode adapted to be placed on and deliver electrical signal to a first nerve or organ;
optionally a second electrode adapted to be placed on and deliver electrical signal to a second nerve or organ;
an implantable pulse generator operably connected to the first and/or the second electrode, wherein the implantable pulse generator comprises a power module and a programmable therapy delivery module, wherein the programmable therapy delivery module is configured to deliver at least one therapy program comprising a first therapy program and optionally a second therapy program, wherein the first therapy program comprises a first electrical signal treatment applied to the first nerve or organ through the first electrode, wherein the second therapy program comprises a second electrical signal treatment applied to the second nerve or organ through the second electrode, and wherein the first and/or the second electrical signal are each configured to initiate activity on the first and/or the second nerve or organ respectively, and wherein the activity is a neural stimulation or a neural block; and
an external component comprising a communication system and a programmable storage and communication module, wherein programmable storage and communication module are configured to store the at least one therapy program and to communicate the at least one therapy program to the implantable pulse generator.
   2. The system of clause 1, wherein the first and/or the second electrical signal are each independently configured to upregulate or down-regulate activity respectively on the first and/or second target nerve or organ.
   3. The system of any one of clauses 1-2, wherein the first and the second electrical signals are applied concurrently, or simultaneously, or intermittently, or during substantially the same times, or during substantially different times, or in a coordinated fashion.
   4. The system of any one of clauses 1-3, wherein the first and/or the second electrical signal treatments are each continuously applied to the first target nerve or organ and/or the second target nerve or organ respectively.
   5. The system of any one of clauses 1-4, further comprising a glucose sensor configured to continuously monitor plasma glucose of the subject, wherein the glucose sensor is operably connected to the implantable pulse generator and the external component.
   6. The system of any one of clauses 1-5, wherein the glucose sensor is configured to detect an increase or decrease of plasma glucose from a pre-determined threshold level.
   7. The system of clause 6, wherein the implantable pulse generator is triggered to deliver the first and/or the second electrical treatment when the plasma glucose of the subject is of or below a first pre-determined threshold, and wherein the implantable pulse generator ceases to deliver the first and/or the second electrical treatment when the plasma glucose of the subject is of or above a second pre-determined threshold.
   8. The system of any one of clauses 1-7, wherein the first nerve or organ and the second nerve or organ are each independently selected from the group consisting of the vagus nerve, anterior vagus nerve, posterior vagus nerve, hiatus on posterior nerve, hepatic branch of vagus nerve, celiac branch of vagus nerve, splanchnic nerve, renal nerve, renal artery, sympathetic nerves, baroreceptors, glossopharyngeal nerve, duodenum, jejunum, ileum, small bowel, colon, stomach, esophagus, liver, spleen, pancreas, and combinations thereof.
   9. The system of any one of clauses 1-8, wherein the first and/or the second electrical signals each have an on time and an off time, wherein the off time is selected to allow at least a partial recovery of the activity of the first and/or the second nerve or organ.
   10. The system of clauses 9, wherein the on time is configured to commence upon the detection of plasma glucose level of or below about 50 mg/dL, of or below about 60 mg/dL, of or below about 70 mg/dL, of or below about 80 mg/dL.
   11. The system of any one of clauses 1-10, wherein the first and/or the second electrical signal treatment are configured to increase the plasma glucose level by at least about 5 mg/dL in about 10 minutes.
   12. The system of any one of clauses 1-11, wherein the first and/or the second electrical signal treatment are configured to increase the plasma glucose level by at least about 10 mg/dL in about 20 minutes.
   13. The system of any one of clauses 1-12, wherein the first and/or the second electrical signal treatment are configured to increase the plasma glucose level by at least about 20 mg/dL in about 30 minutes.
   14. The system of any one of clauses 1-13, wherein the first electrical signal has a frequency of about 0.01 Hz to about 200 Hz.
   15. The system of any one of clauses 1-13, wherein the first electrical signal has a frequency of about 200 Hz to about 10 k Hz.
   16. The system of any one of clauses 1-15, wherein the second electrical signal has a frequency of about 0.01 Hz to about 200 Hz.
   17. The system of any one of clauses 1-15, wherein the second electrical signal has a frequency of about 200 Hz to about 10 k Hz.
   18. The system of any one of clauses 1-13, wherein the first electrical signal has a frequency of about 0.01 Hz to about 200 Hz, and wherein the second electrical signal has a frequency of about 200 Hz to about 10 k Hz.
   19. The system of any one of clauses 1-18, wherein the first electrical signal and/or the second electrical signal each independently comprise a signal pattern, wherein each signal pattern comprises a pulse having a pulse width from about 0.1 microseconds to about 10,000 microseconds.
   20. The system of any one of clauses 1-19, wherein the pulse of the first and/or the second electrical signal is monophasic pulse, or biphasic pulse, or combinations thereof.
   21. The system of any one of clauses 1-20, wherein the first and/or the second electrical signal each independently have an on time of about 30 seconds to about 30 minutes.
   22. The system of any one of clauses 1-21, wherein the first and/or the second electrical signal each independently have a current amplitude in a range from about 0.01 mAmps to about 20 mAmps.
   23. The system of any one of clauses 1-22, wherein the first and/or the second electrical signal each independently comprise an abrupt start of pulses, or a ramp up of current/voltage amplitude, or a ramp up of frequency, or a ramping up of pulse widths, or combination thereof at or near initiation of applying the first and/or the second electrical signal.
   24. The system of any one of clauses 1-23, wherein the first and/or the second electrical signal treatments are configured to be applied intermittently multiple times in a day and over multiple days, wherein the first and/or the second electrical signal each have a frequency selected to upregulate activity on the first nerve or organ and has an on time and an off time, wherein the off time is selected to allow at least a partial recovery of the activity of the first nerve or organ.
   25. The system of any one of clauses 1-24, wherein the programmable storage and communication module are configured to store and communicate more than one therapy program, wherein each therapy program is different from one another, and is configured to be selected for communication.
   26. The system of any one of clauses 1-25, further comprising a transmitter operably connected to the glucose sensor, wherein the transmitter is configured to communicate data generated by the glucose sensor to an external communication device.
   27. The system of any one of clauses 1-26, wherein the communication system is selected from a group consisting of an antenna, blue tooth technology, radio frequency, Wi-Fi, light, sound and combinations thereof, and wherein the communication system is configured to communicate parameters of the at least one therapy program to an external communication device.
   28. A method of treating hypoglycemia in a subject, the method comprising:
applying a first electrical signal to a first nerve or organ of the subject using the system of clause 1, wherein the first electrical signal initiates a neural stimulation or a neural block; and
optionally applying a second electrical signal to a second nerve or organ of the subject using the system of clause 1, wherein the second electrical signal initiates a neural stimulation or a neural block.
   29. The method of clause 28, wherein the first and the second electrical signals are applied concurrently, or simultaneously, or intermittently, or during substantially the same times, or during substantially different times, or in a coordinated fashion.
   30. The method of any one of clauses 28-29, wherein the first and/or the second electrical signal are configured to increase plasma glucose of the subject by at least about 5 mg/dL in about 10 minutes.
   31. The method of any one of clauses 28-30, wherein the first and/or the second electrical signal treatment are configured to increase the plasma glucose level by at least about 10 mg/dL in about 20 minutes.
   32. The system of any one of clauses 28-31, wherein the first and/or the second electrical signal treatment are configured to increase the plasma glucose level by at least about 20 mg/dL in about 30 minutes.
   33. The method of any one of clauses 28-32, wherein the first and/or the second electrical signal are each applied continuously during an on time followed by an off time during which the signal is not applied to the nerve or organ.
   34. The method of any one of clauses 28-33, wherein the on times are applied multiple times per day when plasma glucose level is of or below about 50 mg/dL, of or below about 60 mg/dL, of or below about 70 mg/dL, of or below about 80 mg/dL.
   35. The method of any one of clauses 28-34, wherein the off times are applied multiple times per day when plasma glucose level is of or above about 80 mg/dL, of or above about 90 mg/dL, of or above about 100 mg/dL, of or above about 110 mg/dL.
   36. The method of any one of clauses 28-35, wherein the first electrical signal has a frequency of about 0.01 Hz to about 200 Hz.
   37. The method of any one of clauses 28-35, wherein the first electrical signal has a frequency of about 200 Hz to about 10 k Hz.
   38. The method of any one of clauses 28-37, wherein the second electrical signal has a frequency of about 0.01 Hz to about 200 Hz.
   39. The method of any one of clauses 28-37, wherein the second electrical signal has a frequency of about 200 Hz to about 10 k Hz.
   40. The method of any one of clauses 28-35, wherein the first electrical signal has a frequency of about 0.01 Hz to about 200 Hz, and wherein the second electrical signal has a frequency of about 200 Hz to about 10 k Hz.
   41. The method of any one of clauses 28-40, wherein the first nerve or organ and the second nerve or organ are independently selected from the group consisting of the vagus nerve, anterior vagus nerve, posterior vagus nerve, hiatus on posterior nerve, hepatic branch of vagus nerve, celiac branch of vagus nerve, splanchnic nerve, renal nerve, renal artery, sympathetic nerves, baroreceptors, glossopharyngeal nerve, duodenum, jejunum, ileum, small bowel, colon, stomach, esophagus, liver, spleen, pancreas, and combinations thereof.
   42. The method of any one of clauses 28-41, wherein the first nerve or organ and the second nerve or organ are different.
   43. The method of any one of clauses 28-42, wherein the first electrical signal is applied on a hepatic branch of a vagus nerve or an anterior vagus nerve central to a branching point of a hepatic nerve.
   44. The method of any one of clauses 28-42, wherein the first electrical signal is applied on a celiac branch of a vagus nerve or an anterior vagus nerve central to a branching point of a celiac nerve.
   45. The method of any one of clauses 28-42, wherein the first electrical signal is applied to liver, pancreas or both.
   46. The method of any one of clauses 28-45, wherein the second electrical signal is applied to a splanchnic nerve or a celiac branch of a vagus nerve, or pancreas.
   47. The method of any one of clauses 28-45, wherein the second electrical signal is not involved in the method.
   48. The method of any one of clauses 28-47, further comprising administering an agent that improves glucose control.
   49. The method of clause 48, wherein the agent decreases the amount of insulin and/or decreases the sensitivity of cells to insulin.
   50. A method of making a system for treating hypoglycemia in a subject, the method comprising:
connecting a first electrode to an implantable pulse generator and placing the first electrode to a first nerve or organ;
optionally connecting a second electrode to the implantable pulse generator and placing the second electrode to a second nerve or organ;
configuring a programmable therapy delivery module of the implantable pulse generator to deliver at least one therapy program comprising a first electrical signal treatment and optionally a second electrical signal treatment, wherein the first electrical signal treatment is configured to be applied to the first nerve or organ through the first electrode, and the second electrical signal treatment is configured to be applied to the second nerve or organ through the second electrode, and wherein the first and/or the second electrical signal each initiate a neural stimulation or a neural block; and
configuring a programmable storage and communication module of an external component to store the at least one therapy program and to communicate the at least one therapy program to the implantable pulse generator.
   51. The method of clause 50, wherein the first and/or the second electrical signal each have a frequency selected to initiate activity respectively on the first and/or the second target nerve or organ, and wherein the activity is an upregulation or down-regulation of neural activity.
   52. The method of any one of clauses 50-51, further comprising connecting a glucose sensor to the implantable pulse generator; and monitoring or detecting plasma glucose level of the subject using the glucose sensor.
   53. The method of any one of clauses 50-52, further comprising configuring a communication system of the external component to communicate parameters of the at least one therapy program to an external communication device.
   54. The method of any one of clauses 50-53, further comprising connecting a transmitter to the glucose sensor to communicate data generated by the glucose sensor to an external communication device.
   55. The method of any one of clauses 50-54, wherein the first and/or the second electrode are placed on the nerve or organ via a laparoscopic approach.
   56. A system for treating hypoglycemia in a subject, the system comprising:
at least one electrode adapted to be placed on and deliver electrical signal to a nerve or organ of the subject;
an implantable pulse generator operably connected to the at least one electrode, wherein the implantable pulse generator comprises a power module and a programmable therapy delivery module, wherein the programmable therapy delivery module is configured to deliver at least one therapy program, wherein the at least one therapy program comprises at least one electrical signal treatment applied to the nerve or organ through the at least one electrode,
an external component comprising a communication system and a programmable storage and communication module, wherein programmable storage and communication module are configured to store the at least one therapy program and to communicate the at least one therapy program to the implantable pulse generator, and
a glucose sensor operably connected and to and in communication with the implantable pulse generator and the external component, wherein the glucose sensor is configured to continuously monitor plasma glucose of the subject and to detect an increase or decrease of plasma glucose from a pre-determined threshold level,
wherein, the implantable pulse generator is triggered to deliver the at least one electrical signal treatment when the plasma glucose of the subject is of or below a first pre-determined threshold, and wherein the implantable pulse generator ceases to deliver the at least one electrical signal treatment when the plasma glucose of the subject is of or above a second pre-determined threshold,
wherein the at least one electrical signal treatment is configured to initiate neural stimulation on the nerve or organ of the subject.
   57. The system of clause 56, wherein the nerve or organ is selected from the group consisting of the vagus nerve, anterior vagus nerve, posterior vagus nerve, hiatus on posterior nerve, hepatic branch of vagus nerve, celiac branch of vagus nerve, splanchnic nerve, renal nerve, renal artery, sympathetic nerves, baroreceptors, glossopharyngeal nerve, duodenum, jejunum, ileum, small bowel, colon, stomach, esophagus, liver, spleen, pancreas, and combinations thereof.
   58. The system of any one of clauses 56-57, wherein the nerve is posterior vagus nerve (PVN) of the subject or the celiac vagus nerve branch of the PVN.
   59. The system of any one of clauses 56-58, wherein the electrical signal has an on time and an off time, wherein the off time is selected to allow at least a partial recovery of the activity of the nerve or organ.
   60. The system of clause 59, wherein the on time is configured to commence upon the detection of plasma glucose level of or below about 50 mg/dL, of or below about 60 mg/dL, of or below about 70 mg/dL, of or below about 80 mg/dL in the subject.
   61. The system of any one of clauses 59-60, wherein the on time is about 30 seconds to about 30 minutes.
   62. The system of any one of clauses 56-61, wherein the at least one electrical signal treatment comprises an electrical signal pattern having a frequency from about 1 Hz to about 200 Hz, or from about 1 Hz to about 50 Hz, or from about 1 Hz to about 20 Hz, or from about 1 Hz to about 10 Hz, or from about 1 Hz to about 5 Hz, or from about 1 Hz to about 2 Hz.
   63. The system of any one of clauses 56-62, wherein the electrical signal pattern has a pulse width from about 0.1 microseconds to about 10 microseconds in about 0.1 microseconds steps.
   64. The system of any one of clauses 56-63, wherein the electrical signal pattern has a pulse amplitude from about 0.1 mA to about 12 mA in about 0.1 mA steps.
   65. The system of any one of clauses 56-64, wherein the pulse of the at least one electrical signal is monophasic pulse, or biphasic pulse, or combinations thereof.
   66. The system of any one of clauses 56-65, wherein the at least one electrical signal further comprises an abrupt start of pulses, or a ramp up of current/voltage amplitude, or a ramp up of frequency, or a ramping up of pulse widths, or combination thereof at or near initiation of applying the electrical signal.
   67. The system of any one of clauses 56-66, wherein at least one electrical signal treatment is configured to be applied intermittently multiple times in a day and over multiple days.
   68. The system of any one of clauses 56-67, wherein the at least one electrical signal treatment is configured to causes increase of the plasma glucose of the subject by at least about 5 mg/dL in about 10 minutes, or by at least about 10 mg/dL in about 20 minutes, by at least about 20 mg/dL in about 30 minutes, or by at least about 30 mg/dL in about 45 minutes, or by at least about 40 mg/dL in about 60 minutes.
   69. The system of any one of clauses 56-68, wherein the at least one electrical signal treatment is configured to cause an increase of glucagon secretion in the subject.
   70. The system of any one of clauses 56-69, wherein application of the at least one electrical signal treatment is configured to cause an decrease of insulin secretion in the subject.
   71. The system of any one of clauses 56-70, further comprising a transmitter operably connected to the glucose sensor, wherein the transmitter is configured to communicate data generated by the glucose sensor to an external communication device.
   72. The system of any one of clauses 56-71, wherein the communication system is selected from a group consisting of an antenna, blue tooth technology, radio frequency, Wi-Fi, light, sound and combinations thereof, and wherein the communication system is configured to communicate parameters of the at least one therapy program to an external communication device.
   73. A method of treating hypoglycemia in a subject in need thereof, the method comprising:
applying the at least one electrical signal treatment to a posterior vagus nerve (PVN) of a subject or the celiac vagus nerve branch of the PVN of the subject using the system of any one of clauses 56-72.
   74. The method of clause 73, wherein the electrical signal pattern applied to the has a frequency from about 1 Hz to about 20 Hz, a pulse width from about 0.1 microseconds to about 10 microseconds in about 0.1 microseconds steps, and a pulse amplitude from about 0.1 mA to about 12 mA in about 0.1 mA steps.
   75. The method of any one of clauses 73-74, wherein application of the at least one electrical signal treatment causes increase of the plasma glucose of the subject by at least about 5 mg/dL, at least about 10 mg/dL, at least about 20 mg/dL, at least about 30 mg/dL, at least about 40 mg/dL, at least about 50 mg/dL, at least about 60 mg/dL, at least about 70 mg/dL, at least about 80 mg/dL, at least about 90 mg/dL, or at least about 100 mg/dL, in about 60 minutes.
   76. The method of any one of clauses 73-75, wherein application of the at least one electrical signal treatment causes increase of glucagon secretion in the subject.
   77. The method of any one of clauses 73-76, wherein application of the at least one electrical signal treatment causes decrease of insulin secretion in the subject.
   78. The method of any one of clauses 73-77, further comprising: placing the at least one electrode on the nerve or organ via a laparoscopic approach.

1. A system for treating hypoglycemia in a subject, the system comprising:
a first electrode adapted to be placed on and deliver electrical signal to a first nerve or organ;
optionally a second electrode adapted to be placed on and deliver electrical signal to a second nerve or organ;
an implantable pulse generator operably connected to the first and/or the second electrode, wherein the implantable pulse generator comprises a power module and a programmable therapy delivery module, wherein the programmable therapy delivery module is configured to deliver at least one therapy program comprising a first therapy program and optionally a second therapy program, wherein the first therapy program comprises a first electrical signal treatment applied to the first nerve or organ through the first electrode, wherein the second therapy program comprises a second electrical signal treatment applied to the second nerve or organ through the second electrode, and wherein the first and/or the second electrical signal are each configured to initiate activity on the first and/or the second nerve or organ respectively, and wherein the activity is a neural stimulation or a neural block; and
an external component comprising a communication system and a programmable storage and communication module, wherein programmable storage and communication module are configured to store the at least one therapy program and to communicate the at least one therapy program to the implantable pulse generator.

2. The system of claim 1, wherein the first and/or the second electrical signal are each independently configured to upregulate or down-regulate activity respectively on the first and/or second target nerve or organ.

3. The system of any one of claim 1, wherein the first and the second electrical signals are applied concurrently, or simultaneously, or intermittently, or during substantially the same times, or during substantially different times, or in a coordinated fashion.

4. The system of any one of claim 1, wherein the first and/or the second electrical signal treatments are each continuously applied to the first target nerve or organ and/or the second target nerve or organ respectively.

5. The system of any one of claim 1, further comprising a glucose sensor configured to continuously monitor plasma glucose of the subject, wherein the glucose sensor is operably connected to the implantable pulse generator and the external component.

6. The system of any one of claim 1, wherein the glucose sensor is configured to detect an increase or decrease of plasma glucose from a pre-determined threshold level.

7. The system of claim 6, wherein the implantable pulse generator is triggered to deliver the first and/or the second electrical treatment when the plasma glucose of the subject is of or below a first pre-determined threshold, and wherein the implantable pulse generator ceases to deliver the first and/or the second electrical treatment when the plasma glucose of the subject is of or above a second pre-determined threshold.

8. The system of any one of claim 1, wherein the first nerve or organ and the second nerve or organ are each independently selected from the group consisting of the vagus nerve, anterior vagus nerve, posterior vagus nerve, hiatus on posterior nerve, hepatic branch of vagus nerve, celiac branch of vagus nerve, splanchnic nerve, renal nerve, renal artery, sympathetic nerves, baroreceptors, glossopharyngeal nerve, duodenum, jejunum, ileum, small bowel, colon, stomach, esophagus, liver, spleen, pancreas, and combinations thereof.

9. A method of treating hypoglycemia in a subject, the method comprising: applying a first electrical signal to a first nerve or organ of the subject using the system of claim 1, wherein the first electrical signal initiates a neural stimulation or a neural block; and optionally applying a second electrical signal to a second nerve or organ of the subject using the system of claim 1, wherein the second electrical signal initiates a neural stimulation or a neural block.

10. The method of claim 9, wherein the first and the second electrical signals are applied concurrently, or simultaneously, or intermittently, or during substantially the same times, or during substantially different times, or in a coordinated fashion.

11. The method of any one of claim 9, wherein the first and/or the second electrical signal are configured to increase plasma glucose of the subject by at least about 5 mg/dL in about 10 minutes.

12. The method of any one of claim 9, wherein the first and/or the second electrical signal treatment are configured to increase the plasma glucose level by at least about 10 mg/dL in about 20 minutes.

13. The system of any one of claim 9, wherein the first and/or the second electrical signal treatment are configured to increase the plasma glucose level by at least about 20 mg/dL in about 30 minutes.

14. The method of any one of claim 9, wherein the first and/or the second electrical signal are each applied continuously during an on time followed by an off time during which the signal is not applied to the nerve or organ.

15. The method of any one of claim 9, wherein the on times are applied multiple times per day when plasma glucose level is of or below about 50 mg/dL, of or below about 60 mg/dL, of or below about 70 mg/dL, of or below about 80 mg/dL.

16. The method of any one of claim 9, wherein the off times are applied multiple times per day when plasma glucose level is of or above about 80 mg/dL, of or above about 90 mg/dL, of or above about 100 mg/dL, of or above about 110 mg/dL.

17. The method of any one of claim 9, wherein the first electrical signal has a frequency of about 0.01 Hz to about 200 Hz.

18. The method of any one of claim 9, wherein the first electrical signal has a frequency of about 200 Hz to about 10 k Hz.

19. The method of any one of claim 9, wherein the second electrical signal has a frequency of about 0.01 Hz to about 200 Hz.

20. The method of any one of claim 9, wherein the second electrical signal has a frequency of about 200 Hz to about 10 k Hz.
